(19) United States
(12) Patent Application Publication
(10) Pub. No.: US 2002/0156361 A1
(43) Pub. Date: Oct. 24, 2002

(54) Positioning template for implanting a substance into a patient

(76) Inventors: Youri Popowski, Geneva (CH); Giovanni Leo, Chene Bougeries (CH)

Correspondence Address: Michael J. McGrath, CROMPTON, SEAGER & TUFTE, LLC, Suite 895, 331 Second Avenue South, Minneapolis, MN, 55401-2246, US

(21) Appl. No.: 10/171,332

(22) Filed: Jun. 12, 2002

Related U.S. Application Data
(63) Continuation-in-part of application No. 09/692,583, filed on Oct. 19, 2000.

(51) Int. Cl. A61B 5/05

(52) U.S. Cl. 600/407

(57) ABSTRACT

Template grid and method of implanting or delivering substances into a living being. The template grid may include a planar surface and a plurality of holes disposed within the planar surface adapted for receiving a plurality of individual objects. Additionally, a first individual object can be attached to a hole independently of a second individual object. The template grid may be used in conjunction with an imaging technique.

### CROSS-REFERENCE TO RELATED APPLICATION

This application is a continuation-in-part of co-pending U.S. patent application Ser. No. 09/692,583, filed Oct. 19, 2000 entitled BRACHYTHERAPY POSITIONING TEMPLATE, the entire disclosure of which is herein incorporated by reference.

### FIELD OF THE INVENTION

This invention generally relates to a positioning template grid for medical implantation procedures. More specifically, the invention relates to a positioning template for implantation and/or delivery of substances into a patient.

### BACKGROUND OF THE INVENTION

The American Cancer Society estimates that approximately 1 in every 4 deaths in the United States is due to cancer. This translates to an enormous amount of people who are affected either directly or indirectly by cancer. Because of the numbers of people involved there has been a great deal of interest in improving treatment options for people suffering from cancer.

Historically, cancer treatment has primarily included surgery, chemotherapy, and radiation treatment. Surgical removal of a tumor is probably the most straightforward way to eliminate tumors. However, tumor removing surgeries can be highly invasive. Further, since it may be advantageous to remove an amount of tissue slightly larger than the tumor in order to be sure that the entire tumor is removed, surgical excision of tumors can be limited by the afflicted region. For example, when tumors are confined to the brain it can be difficult to completely remove an entire tumor without removing any healthy brain tissue.

Many efficacious cancer chemotherapy drugs exist that can kill or slow the growth of tumors. Although effective, cancer chemotherapy may be limited by an inability to act at a specific target location. Moreover, cancer chemotherapeutics are commonly associated with undesirable side effects. For these reasons, traditional or general routes of administering cancer chemotherapeu tics may not be appropriate for the treatment of some cancers.

Radiation treatment can be used as a cancer treatment and is typically directed to a specific tumor or cancerous region. This can limit side effects since only the affected region is irradiated. However, if the target region is deep within the body, or near a particularly sensitive body region or organ, it may be difficult to use radiation without affecting healthy tissue.

A large body of research has focused on improving the above methods of cancer treatment as well as developing new or alternative treatment strategies. Accordingly, there is an ongoing need to develop improved cancer therapies that are, for example, more efficacious, more specific, and have less side effects.

### BRIEF SUMMARY

The present invention pertains to template grids for implanting or delivering substances into a patient. In at least some embodiments, a template grid may be used for implantation of substances to facilitate an appropriate diagnostic or therapeutic medical procedure. Some examples of appropriate medical procedures and/or medical substances include cancer diagnosis; drug delivery or implantation including delivery of antibiotics and anti-viral drugs; cancer treatment including chemotherapy, brachytherapy (for example, treatment of pelvic tumors, prostate tumors, gynecological cancers, breast cancer, etc.), implantation or delivery of a radioactive material (e.g., radioactive and/or ferromagnetic seed implantation); implantation and delivery of gene therapy substances; implantation and delivery of oligopeptide and oligonucleotides; local laser treatment; and treatment of prostate benign hypertrophy. In some embodiments, the template grid may further comprise one or more delivery members, one or more securing members, and one or more reference devices.

### BRIEF DESCRIPTION OF THE DRAWINGS

FIG. 1A illustrates an example embodiment of a template grid viewed from a generally planar surface;

FIG. 1B illustrates an example embodiment of a template grid viewed from a second surface;

FIG. 1C illustrates an example embodiment of a template grid viewed from a side surface;

FIG. 1D illustrates an example embodiment of a template grid viewed across a transverse plane;

FIG. 2 is a delivery member to facilitate medical procedure;

FIG. 3 is a securing member capable of attaching to the generally planar surface of the template grid;

FIG. 4 is an alternate embodiment of a securing member capable of attaching to a template grid;

FIG. 5 illustrates an example embodiment of a screwdriver adapted for driving securing members;

FIG. 6 illustrates an example embodiment of a trocard needle;

FIG. 7 is a perspective view of the template grid; and

FIG. 8 is a plan view of the template grid for use in gynecological implantation.

### DETAILED DESCRIPTION

The following description should be read with reference to the drawings wherein like reference numerals indicate like elements throughout the several views. The detailed description and drawings illustrate example embodiments of the claimed invention.

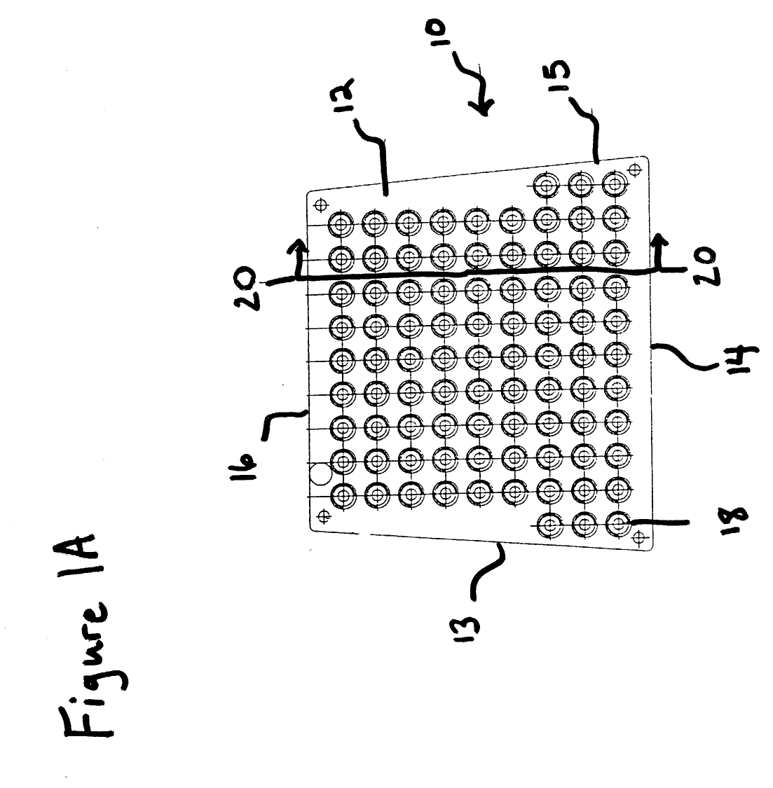

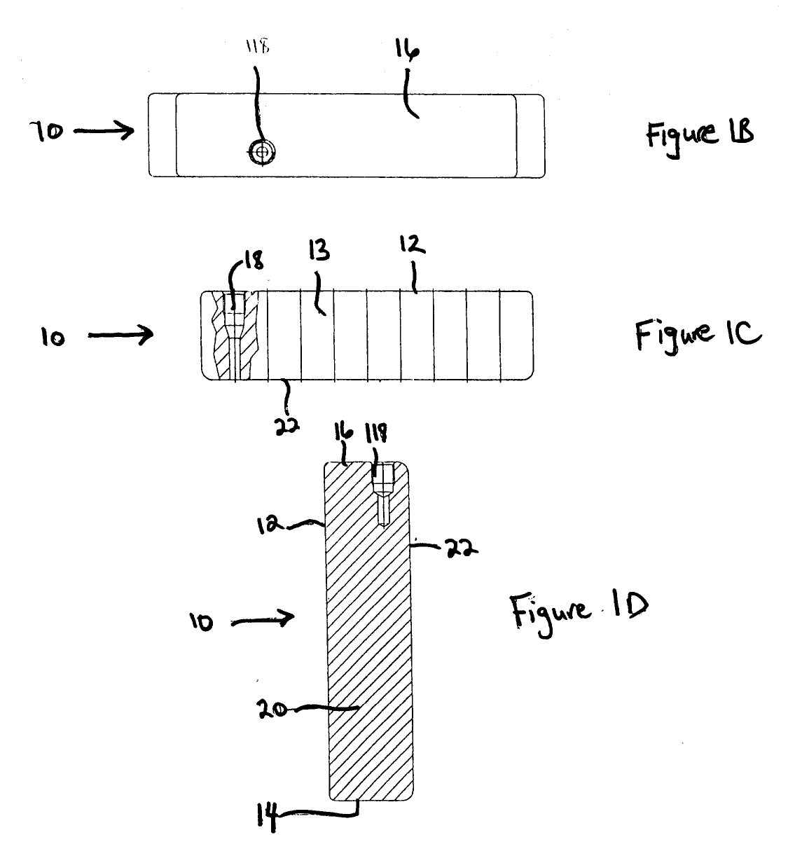

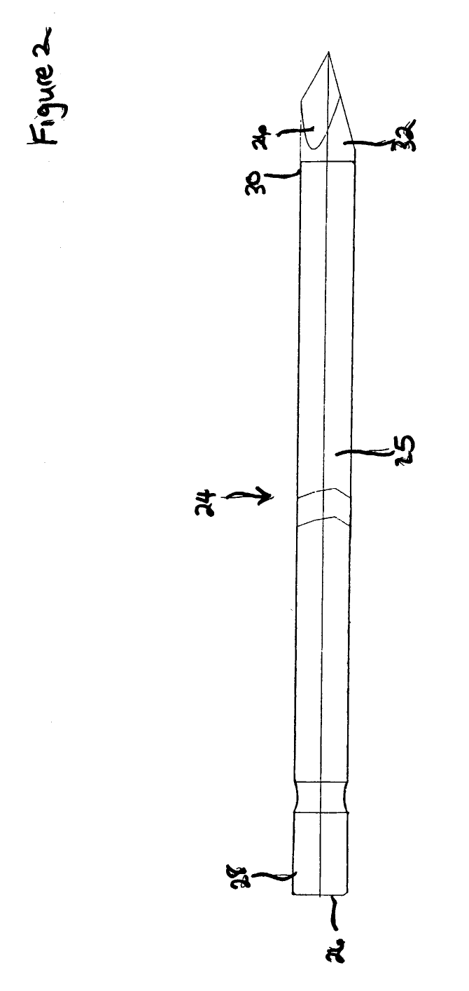

FIG. 1A illustrates an example of a template grid 10. Template grid 10 may include a generally planar surface 12, a side surface 13, a bottom surface 14, a second side surface 15, a top surface 16, and a plurality of holes 18 disposed within the planar surface 12. The holes 18 are adapted for receiving one or more objects. Objects that can be attached to an individual hole 18 include, but are not limited to, a delivery member such as a needle, a catheter, and a trocard needle; a securing member such as a screw; and a reference device such as one or more infrared reflecting spheres and/or one or more infrared light emitting diodes and the like. Each of these objects are described in more detail below. A transverse cut through generally planar surface 12 of template grid 10 defines transverse plane 20.

Template grid 10 can be manufactured from one or more of a plurality of materials. For example, template grid 10 may be manufactured of a polymer, metal, or metal-polymer composite. Polymers may provide template grid 10 with desirable properties. For example, polymers may provide good mechanical properties and high sterilization compatibility. In general, template grid 10 as well as other devices described herein are manufactured of materials compatible with the intended imaging technique to be used with the appropriate intervention. For example, if template grid 10 is being used in conjunction with magnetic resonance imaging (MRI), it may be desirable to manufacture template grid 10 (and other devices used therewith) of an MRI compatible material.

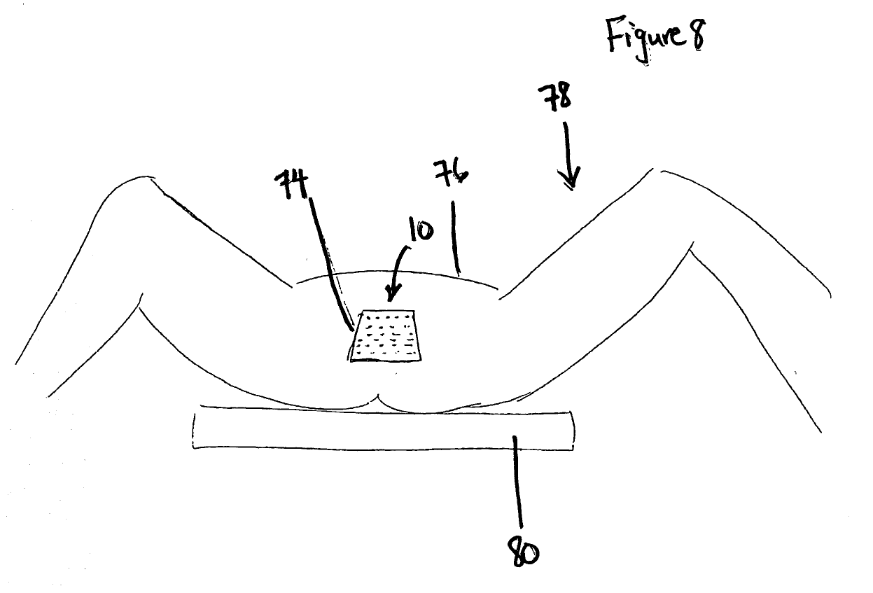

In some embodiments, template grid 10 further comprises an orientation grid disposed within generally planar surface 12 as best seen in FIG. 7. The orientation grid may include a series of letters and numbers listed along generally planar surface 12 of template grid 10. The letters and numbers may be used to uniquely identify the position of each individual hole 18. A person of ordinary skill in the art would be familiar with using an orientation grid according to multiple embodiments of the current invention.

Template grid 10 can be used to facilitate the delivery or implantation of substances into a patient. Traditionally, substances such as drugs are administered orally, by intra-venous infusion, sub-cutaneously, intramuscularly, etc. For example, a chemotherapeutic drug is often administered by intra-venous infusion. Because chemotherapy often involves repeated intra-venous administration of the chemotherapeutic drug, patients often endure repeated puncturing of their veins, which can be traumatic to the patient as well as their veins. In order minimize this trauma, a permanent catheter may be placed under the skin having one end disposed within a vein and another connected to a small box with a membrane. A clinician can then access the vein through the membrane (which may be just under the skin). The treatment strategy of maintaining permanent access to the veins allows a clinician to infuse drugs over a period of days, which has shown efficacy in some cancers (e.g., colorectal cancers). However, this strategy may still be associated with extended hospitalization of the patient.

A more sophisticated treatment strategy has also been developed called loco-regional chemotherapy. According to this technique, a clinician can selectively catheterize an artery that feeds a tumor. The intra-arterial catheter can be left in place for several days for drug delivery, leading to a higher drug concentration in the particular target structure. This selective catheterization and chemotherapy has been used to treat some cancers including, for example, anal, penis, cervical, and uterine cancer. This type of treatment may result in tumor shrinkage, which may suppress the need for surgery or radiation therapy. However, intra-arterial catheters cannot be used with the small box as described above because the risk of thrombosis is much higher in an artery than in a vein. Because of this, the arterial catheter has to be removed after several hours or days, which may limit the efficacy of this mode of treatment.

Many interventions, including cancer treatment, are improved by combining several modes of treatment. By combining treatment strategies, side effects can be lowered (for example, by decreasing the dose of drugs). In at least some embodiments, template grid 10 can be used in conjunction with and/or to facilitate the above catheterization strategies. In addition to chemotherapy, template grid 10 can also be used to facilitate other cancer therapies such as radiation therapy, brachytherapy (radioactive seed implantation), surgery, etc. For example, the addition to or the replacement of radioactive seeds by chemotherapeutic implants via template grid 10 could improve the overall efficacy of the intervention. In some instances, the success rate of prostate cancer treatment by permanent radioactive seed implants may approach 85%. It is believed that by adding local chemotherapy implants using template grid 10 to seed implantation, the success rate could go up to 95% or more, without increasing the toxicity of the treatment. The addition of the chemotherapy implantation could also allow a decrease in the dose of radiation necessary or tumor control, leading to a decrease in radiation-associated toxicity (e.g., maintained sexual potency after radiotherapy for prostate cancer). Alternatively, template grid 10 can be used instead of the above therapies to facilitate diagnosis or treatment and overcome at least some of the limitations attributed to those therapies.

Template grid 10 may be used for implantation or delivery of substances to facilitate an appropriate diagnostic or therapeutic medical procedure to essentially any part of the body. Some examples of appropriate medical procedures and/or medical substances include cancer diagnosis; drug delivery or implantation including delivery of antibiotics (e.g., for treating prostatitis)and anti-viral drugs; cancer treatment including chemotherapy, brachytherapy (for example, treatment of pelvic tumors, prostate tumors, gynecological cancers, breast cancer, etc.), implantation or delivery of a radioactive material (e.g., radioactive and/or ferromagnetic seed implantation); implantation and delivery of gene therapy substances; implantation and delivery of oligopeptide and oligonucleotides; local laser treatment; deposition of diagnostic substances in order to allow the detection of the sentinel nodes; repair of a fibrosis or a necrosis (e.g., by delivering one or more growth factors); injection of growth factors for bone and cartilage recovery (e.g. into the hip to treat hip osteoporosis and/or to prevent or treat hip fractures); injection into the heart (e.g., to allow muscle recovery after a myocardial infarction); and treatment of prostate benign hypertrophy.

Template grid 10 can be used at essentially any body location. For example, template grid 10 can be used to deliver or implant substances adjacent the pelvic area (including the prostate, rectum, uterus, ovary, etc.); to bones (including the into head of the femur, pelvic bones, etc.); the abdomen (including the intestines, kidneys, liver, stomach, spleen, pancreas, gall bladder, etc.); the central nervous system (including into brain tumors); the thoracic cavity (including the heart and lungs); the head and neck (including the esophagus); and any soft tissue (including muscle, fat, connective tissue, etc.).

In at least some embodiments, template grid 10 may be used to implant or deliver a substance interstitially. Delivery or implantation interstitially is understood to mean deposition of a material within a tissue. For example, interstitial delivery may be within a tumor mass, within an organ, etc. Interstitial delivery and/or implantation may be a desirable alternative or combination to the above therapies because it allows specific targeting of a substance to an area of interest and the ability to treat the whole volume of the target area.

Substances that may be delivered and/or implanted in conjunction with template grid 10 can include pharmacological agents and/or drugs (including chemotherapeutic, antibiotic, and anti-viral drugs). However, it can be appreciated that essentially any substance may be delivered and/or implanted in conjunction with template grid 10. The substance may be used in any one of a number of different shapes or forms including, for example, a liquid, paste, stick, mesh, a slow release form (including a slow release form of the aforementioned forms), etc. A slow release form may allow longer duration treatment that may be desirable for some treatment strategies, for example, applications where the substance is targeted to a specific location for treatment over a period of time. Additionally, a slow release form may give a clinician greater control and help reduce some of the side effects commonly associated with drugs delivered intra-arterially or intravenously. Differing slow release formulations may be used that elute the substance over a period of days, weeks, or months and generally give the clinician greater control. Moreover, using a paste, stick, or mesh form may allow the substance to be visualized (e.g., by MRI, CT scan, ultrasound, 3-dimensional ultrasound, infrared imaging, etc.) and/or monitored in three-dimensional space during and after delivery or implantation. Alternatively, the substance may encapsulated or be in the shape of a liposome, solid lipid nanosphere, etc.

Some examples of appropriate substances that may include anti-thrombogenic agents such as heparin, heparin derivatives, urokinase, and PPack (dextrophenylalanine proline arginine chloromethylketone); anti-angiogenesis agents; anti-proliferative agents such as enoxaprin, angiopeptin, or monoclonal antibodies capable of blocking smooth muscle cell, hirudin, acetylsalicylic acid, and new generation anti-proliferative substances including anti-tyrosine kinase agents; anti-inflammatory agents such as dexamethasone, prednisolone, corticosteroids including corticosterone, budesonide, estrogen, sulfasalazine, non-steroidal anti-inflammatory agents, and mesalamine; contrast agents; radioactive and non-radioactive diagnostic and/or therapeutic agents including methylene blue, technetium-99 m, and 18-flurodeoxyglucose; cytokines; antineoplastic/antiproliferative/anti-miotic agents such as paclitaxel, 5-fluorouracil, cisplatin, vinblastine, vincristine, epothilones, endostatin, angiostatin and thymidine kinase inhibitors; radioactive seeds; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin anticodies, anti-platelet receptor antibodies, aspirin, prostaglandin inhibitors, platelet inhibitors and tick antiplatelet peptides; antibiotics; vitamins; fibrinolytic agents including streptokinase, anistreplace, alteplase, urokinase, tranexamic acid, aprotinin, ethamsylate, and hyaluronic acid; anti-viral agents; substances used for photodynamic therapy including amino levulinic acid; substances for performing local oxidation and/or reduction (with or without the presence of light) including cobalt or iron complexes with substituted phthalocyanines or naphthalocyanine; growth factors and vascular cell growth promotors such as vascular endothelial growth factor, growth hormone, growth factor inhibitors, growth factor receptor antagonists, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; and cholesterol-lowering agents; vasodilating agents; agents which interfere with endogenous vascoactive mechanisms; nucleic acids including DNA, RNA, and anti-sense RNA; peptides and proteins including enzymes, hormones, anti-bodies, tyrosine kinase agents (including anti-tyrosine kinase agents); DNA coding for (and the corresponding proteins) anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin like growth factor, cell cycle inhibitors including CD inhibitors, thymidine kinase (“TK”) and other agents useful for interfering with cell proliferation, and the family of bone morphogenic proteins (“BMP's”) including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, BMP-16, “hedgehog” proteins; derivates of any of the above; and the like.

In some embodiments, the substance may include an energy source that can, for example, be introduced through delivery member 24 or may comprise an alternative delivery member. It has been shown that different types of energies can be used to treat cancerous lesions. Moreover, these different types of energy can potentiate the effect of therapeutic substances delivered inside the living body. Essentially any energy source may be used with template grid 10 for local deposition of energy. For example, the energy source may be a source of heat energy that can be delivered to a target location by passing an infrared device or antenna through hole 18 (for example, in embodiments where the infrared device is the delivery member) and/or delivery member 24, which may or may not be secured by securing member 34, to delivery infrared energy to the target location. Alternatively, the energy may also be emitted by a waveguide, by a radiofrequency device, by a microwave antenna, or by an ultrasonic probe. Each of the above device be used to treat, for example, a tumor and/or could enhance the efficacy of chemotherapy. Additionally, the use of cryotherapy may also be efficacious either alone or in combination with other interventions. For example, a cryotherapy probe could be placed inside a tumor using template grid 10. Therefore, grid 10 can be used not only to deposit a substance, but also to deposit energy such as heat or cold or another kind of energy locally, in order to increase the effect of other therapeutic methods.

In at least some embodiments, holes 18 are adapted and configured to allow multiple, parallel, paths for delivery and/or implantation of the substance. For example, holes 18 may be uniformly arranged in parallel columns so that a clinician can utilize a subset of holes 18 to ensure optimal, homogenous coverage of a target area. Additionally, this arrangement may allow a clinical to deliver and/or implant the substance to encompass essentially the entire target area and, if desired, regions adjacent the target area. It can be appreciated that multiple different arrangements of holes 18 can be used in different embodiments. For example, holes 18 may be arranged in a non-uniform configuration when it is desirable to concentrate delivery within a particular area.

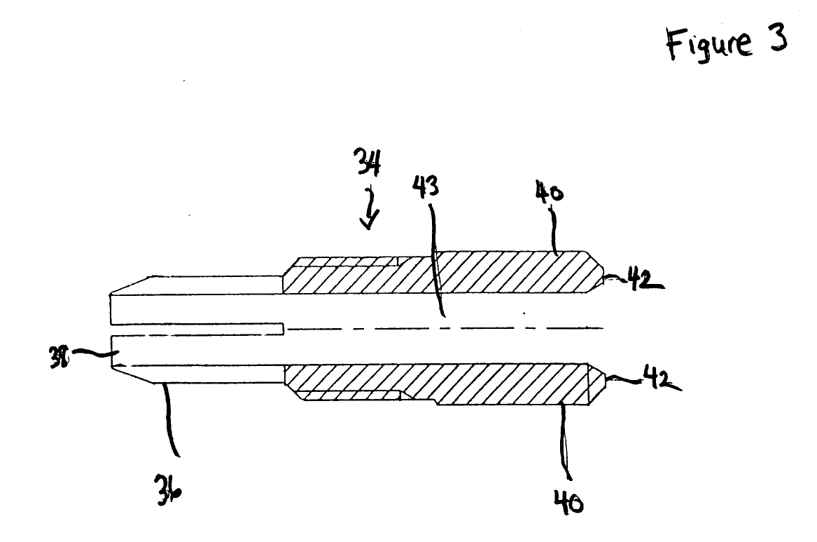

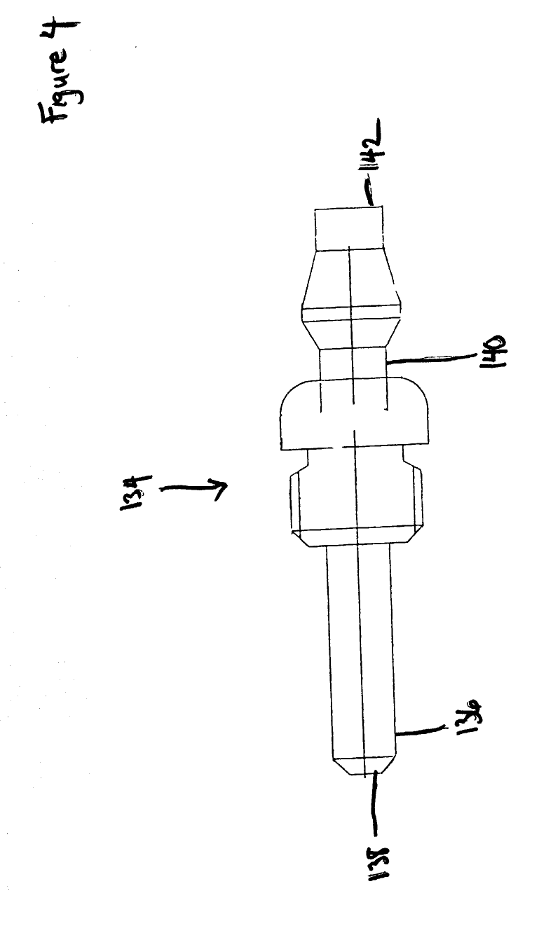

FIGS. 1B-1D illustrate template grid 10 from different perspectives. For example, FIG. 1B shows template grid 10 viewed from top surface 16. In at least some embodiments, top surface 16 of template grid 10 includes at least one hole 118. Hole 118 can be adapted for having a securing member (for example, securing member 34 as best seen in FIG. 3) disposed therein that has a reference device attached thereto. Alternatively, hole 118 can also accommodate any of the other devices described herein such as a delivery member (for example, delivery member 24 as best seen in FIG. 2). FIG. 1C shows template grid 10 viewed from side surface 13 illustrating hole 18 (partially cut away), planar surface 12, and a back surface 22. Similar to what is described above, a number of individual objects may be disposed within hole 18. FIG. 1D shows template grid 10 viewed across transverse plane 20 and showing planar surface 12, bottom surface 14, top surface 16, back surface 22, and hole 118.

In at least some embodiments, template grid 10 can be placed adjacent or against the skin of a patient near an area of interest. An imaging technique (e.g., MRI, CT scanning, ultrasound, 3-dimensional ultrasound, infrared imagining, etc.) may be used to generate a set of images that can provide a clinician with a multiplicity of the potential target areas that could be diagnosed or treated. As described above, diagnosis and/or treatment includes medical implantation and/or delivery of a substance and the imaging technique may also be used to anticipate where the substance is or should be delivered to.

The imaging technique may further comprise an additional technique for coordinating template grid 10 with images generated from the imaging technique. For example, one or more reference devices (best seen in FIG. 7 as reference device 64) may be coupled to template grid 10 that allows template grid 10 to be coordinated with the imaging technique. The physician may then determine, for example, by using a computer and software associated with coordinating template grid 10 (e.g., via the reference device) with the imaging technique, which areas to diagnose or treat. Moreover, coordinating template grid 10 with the imaging technique allows the clinician to anticipate where delivery member 24 (and/or the substance) will go. For example, the clinician may look through the generated images and determine where the substance or a delivery member 24 should arrive or be guided to (i.e., the depth of the implantation). Once validated, in a second step the computer can list the holes and the depth of insertion that should to utilized to guide the substance to the target area. Thus, template grid 10 allows the clinician to deliver a substance to a specific target location, which increases the efficiency, augments the accuracy of the implantation, avoids unnecessary puncturing of the patient, and allows the clinician to treat or diagnose the entire target region. Moreover, the amount of anesthesia required as part of the diagnosis or treatment may be reduced (e.g., from general anesthesia to local anesthesia) or eliminated as a result of the aforementioned benefits.

FIG. 2 is an example of delivery member or needle 24 that can be used for performing medical procedures including the procedures listed above. In at least some embodiments, delivery member 24 comprises a needle. Alternatively, delivery member 24 may include any suitable device capable of passing a substance from or through template grid 10 to the target region such as a tube, catheter, etc. Needle 24 may include tube 25 that defines lumen 26 extending from proximal end 28 to distal end 30 wherein lumen 26 terminates at needle point 32.

Needle 24 can be used to perform implantation and/or delivery and can be constructed of a material compatible with the imaging technique used. In at least some embodiments, needle 24 is constructed of an MRI compatible material such as titanium or Ti—Zr. Titanium and Ti—Zr needles are used for their good mechanical properties as well as CT and MRI compatibility. In alternative embodiments of the invention, needles can be constructed from metals including stainless steel; metal alloys; polymers including polyether-ether ketone (PEEK), nylon (CELCOM), etc.; or any other suitable material.

In some embodiments, delivery member 24 can be used to facilitate an appropriate medical procedure. Needle 24 can be attached to planar surface 12 with a securing member (an example of which is securing member 34 shown in FIG. 3). For example, needle 24 may be attached to planar surface 12 with a screw disposed within hole 18. In some embodiments, needle 24 can be a trocard needle for substance implantation, catheter implantation, or fiber implantation.

Tube 25 and needle point 32 can be constructed of a multiplicity of materials, which may differ from one another. In some embodiments, needle point 32 is constructed of titanium. Alternate embodiments of the invention include a needle point constructed of materials other than titanium. A person of ordinary skill in the art would be familiar with an appropriate material for the manufacturing of a needle point according to multiple embodiments of the current invention. Tube 25 can be constructed of one or more of a multiplicity of materials. For example, tube 25 may be constructed of an MRI compatible material similar to what is described above. Multiple embodiments of the invention include tube 25 manufactured in different sizes. The size of tube 25 includes sizes appropriate for performing medical implantation. For example, tube 25 may be about 180-200 mm in length.

In an alternate embodiment of the invention, needle 32 can be manufactured by combining tube 25 and needle point 32. This embodiment may be desirable when needle point 32 and tube 25 are manufactured from differing materials. A person of ordinary skill in the art would be familiar with the methods for manufacturing a needle according to this embodiment of the invention.

In some embodiments, delivery member 24 can used for permanent or non-permanent implantation or delivery of a substance. For example, delivery member 24 may be left in place for a varying amount of time, for example several hours or days, to allow a medical substance to circulate or diffuse throughout delivery member 24 and the surrounding tissue (or from delivery member 24 into the surrounding tissue). After treatment, delivery member 24 can be removed. Moreover, delivery member 24 can be used in conjunction with an injecting or infusing pump. The pump may allow a clinician to control the amount or rate of delivery of the medical substance through delivery member 24.

Template grid may also be used to deliver substances to, implant substances within, and extract substances (for example bone marrow) from bones. For example, delivery member 24 may be used essentially as described above to deliver or implant a substance. Alternatively, a drill or other suitable device may be passed through delivery member 24 and allowed to generate an opening within the bone. An extracting device (such as a needle, tube, biopsy device, etc.) may then be used to extract bone marrow or other tissue from the bone.

FIG. 3 illustrates a securing member such as a screw 34 capable of being disposed within hole 18 and being releasably attached to planar surface 12 of template grid 10. Screw 34 includes a distal end 36. Distal end 36 includes distal terminus 38 that may be adapted for attaching to a surface of a template grid (i.e., being disposed within hole 18 and being releasably attachable to planar surface 12). Screw 34 further includes proximal end 40 with proximal terminus 42. A driving surface is disposed within proximal terminus 42. In an exemplary embodiment of the invention, screw 34 further comprises lumen 43 that is adapted to allow passage therein of an individual object (e.g., delivery member 24). In at least some embodiments, screw 34 is constructed of an MRI compatible material such as titanium. However, it can be appreciated that screw 34 could also be manufactured from other materials including other metals, metal alloys, polymers, plastics, etc.

Distal end 36 of screw 34 may be conical in shape and/or threaded. The threads of screw 34 may be adapted and configured to mate with threads disposed adjacent holes 18 of planar surface 12. Additionally, hole 18 (and hole 118) may be tapered as best seen in FIG. 1C. According to this embodiment, screw 34 may essentially comprise a collet such that when the cone-shape of distal end 36 is advanced through hole 18, template 10 forces distal end 36 to close as the size of hole 18 decreases and, if screw 34 is driven far enough into hole 18, screw 34 will close so as to essentially block or occlude hole 18. Multiple holes 18 within template 10 can each be blocked with a screw 34. This feature may allow a clinician to substantially prevent unwanted substances from passing through holes 18 at undesired times.

The conical shape near distal end 36 of screw 34 may also be used to fix delivery member 24 in place within hole 18 (and/or planar surface 12 of template grid 10). In at least some embodiments of the invention, screw 34 is adapted to allow passage of an delivery member 24 through lumen 43. When screw 34 is tightened, the conical shape near distal end 36 can shrink against delivery member 24 when screw 34 is driven further into template grid 10. If tightened an appropriate amount, the conical shape near distal end 36 can press against an delivery member 24 so as to fix it in place.

It can be appreciated that in alternate embodiments, securing member 34 may comprise a structure other than a screw. For example, securing member 34 may comprise a clamp, clip, collet, or other suitable device. Additionally, securing member 34 may comprise objects to secure, for example, delivery member 24 or other appropriate structures to template grid 10 at positions other than adjacent hole 18/118. For example, it may be desirable to secure one or more delivery members 24 to side surface 13 (or second side surface 15) if a targeted area is identified that would not practicably be reached through hole 18. Additionally, template grid 10 may include a recess, slot, or guide and securing member 34 may be used to secure, for example, delivery member 24 to any one of these structures.

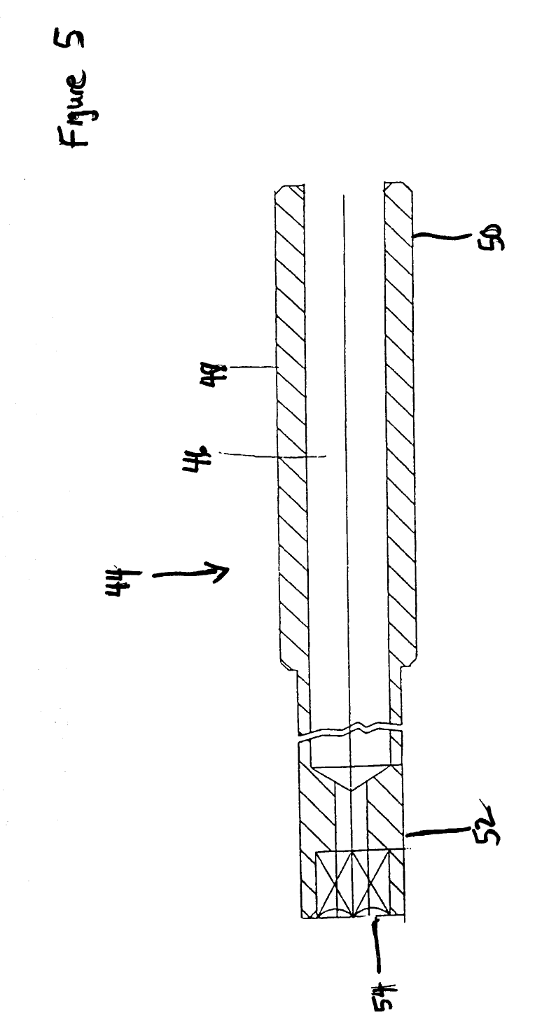

FIG. 4 is an alternate screw 134 capable of attaching to template grid 10 (e.g., hole 18 of planar surface 12 or hole 118). Screw 134 may be adapted for attaching an infrared reflecting sphere or other suitable reference device to a template grid. Screw 134 includes a distal end 136. Distal end 136 includes distal terminus 138 that may be adapted for attaching to generally planar surface 12 of template grid 10. According to an alternate embodiment of the invention, distal terminus 138 is adapted for attaching to top surface 16 of template grid 10. Screw 134 further includes proximal end 140 with proximal terminus 142. The reference device can be attached to proximal terminus 142. A driving surface (e.g., a detent, flange, or the like) may be disposed within proximal end 140. Screw 134 can be made of an MRI compatible material such as titanium, but could be made out of other materials including, but not limited to, metals, metal alloys, polymers, plastic (such as PEEK), etc.

As alluded to above, screw 134 may be adapted to attach to a reference device (best seen in FIG. 7 as reference device 64) to template grid 10. Because template grid 10 may be manufactured of an MRI compatible material, grid 10 is essentially “invisible” to the imaging technique. Thus, reference device 64 can be used to coordinate the position of template grid 10 with the images generated by the imaging technique so that the substance can be accurately delivered to desired location.

In some embodiments, reference device 64 (please see also FIG. 7) may comprise one or more infrared reflecting spheres, for example a set of three reflecting spheres. In order to coordinate reference devices 64 with images generated by the imaging technique (e.g., MRI, CT, ultrasound, 3-D ultrasound, IR, etc.), the set of three infrared reflecting spheres is attached to template grid 10 using screw 134 at top surface 16 and/or planar surface 12. A stereo camera system (e.g., Marconi iPath 200) can then be used to recognize the reflecting sphere configuration and may be able to determine the position of template 10 in respect to the images generated by the imaging technique. More particularly, the system may be used to coordinate the position of template grid 10 with the images generated by the imaging technique so that delivery member 24 can be positioned (for example, in a pre-planned position) and the substance can be accurately delivered to the targeted location. Thus, the system may help anticipate where a needle may pass if implanted into a patient and the final target destination of the substance within the body. In alternate embodiments of the invention, reference device 64 may comprise one or more infrared light emitting diodes (for example, three light emitting diodes) in replacement of the reflecting spheres.

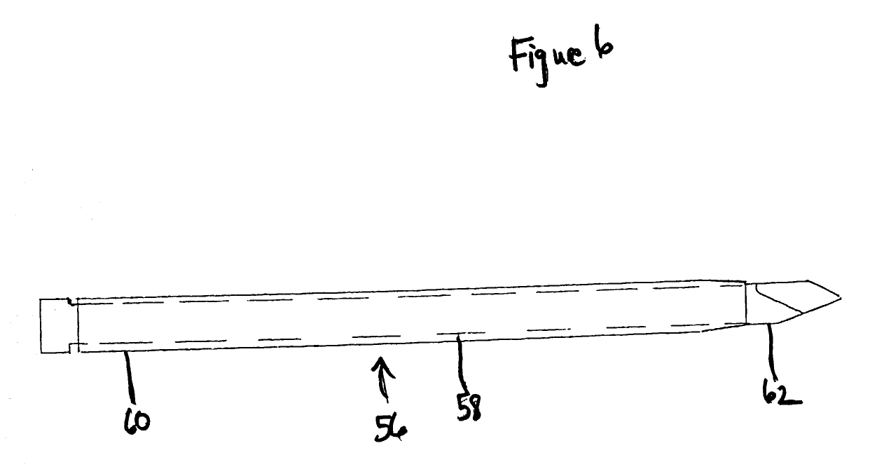

FIG. 5 illustrates a screwdriver 44 adapted for attaching screws 34/134 to template grid 10. Screwdriver 44 may be constructed of an MRI compatible material so as to be insensitive (or invisible) to the MRI magnetic field. Screwdriver 44 further comprises a hole 46 through body 48 of screwdriver 44 to allow screwdriver 44 to pass over, for example, delivery member 24. Screwdriver 44 further comprises proximal end 50, distal end 52, and a driving surface 54 disposed within distal end 52.

Screwdriver 44 may be used to attach screw 34/134 to template grid 10. In some embodiments, delivery member 24 or another suitable structure may be attached to screw 34/134. For example, delivery member 24 may be inserted through lumen 43 disposed within screw 34. The combined delivery member 24 and screw 34 may then be placed into hole 18 disposed with template grid 10. The screw driver 44 can then be placed over delivery member 24 to drive screw 34 into planar surface 12 at hole 18.

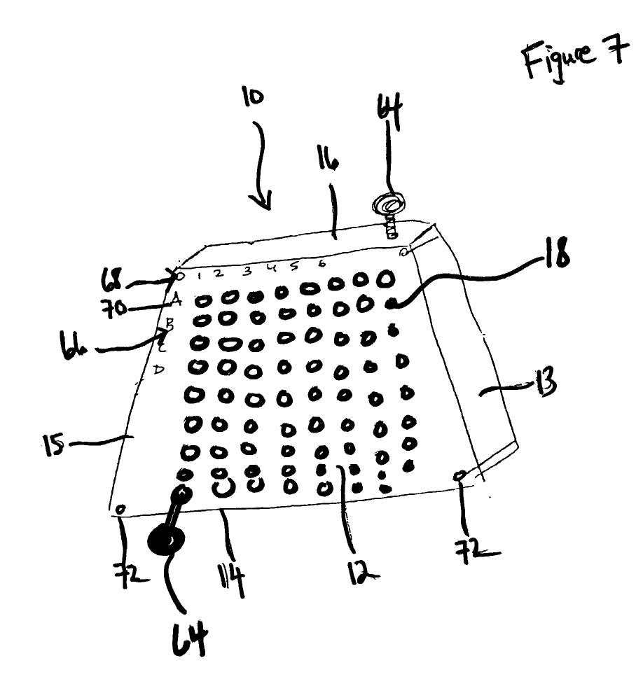

FIG. 6 illustrates an alternative delivery member such as a trocard needle 56. Trocard needle 56 can be used for delivering or implanting a pharmacological agent in a manner similar to what is described above. Trocard needle 56 comprises internal part 58 and external sleeve 60. Internal part 58 is removable from external sleeve 60 and allows for introducing pharmacological agents to an area of interest as described above. Internal part 58 further comprises trocard needle point 62.

FIG. 7 is a perspective view of template grid 10 and illustrating generally planar surface 12, side surface 13, bottom surface 14, side surface 15, top surface 16, and a plurality or holes 18. Top surface 16 may be adapted for attaching objects including one or more reference devices 64, for example, an infrared reflecting sphere as described above. Reference devices 64 may be coupled to template grid 10 at any surface including top surface 16 and/or planar surface 12.

In an exemplary embodiment, template grid 10 further includes orientation grid 66 comprising, for example, number grid 68 and letter grid 70. Orientation grid 66 may be used to uniquely identify positions on template grid 10. In some embodiments, template grid 10 includes small holes 72 that may be used for fixation of template grid 10 to the patient, for example adjacent an area of interest.

In use, a method of using template grid 10 may include the steps of providing template grid 10, placing a patient in a position appropriate for an intended medical procedure, imaging an area of interest of the patient using an imaging technique, positioning template grid 10 adjacent to the area of interest, aligning template grid 10 with images generated by the imaging technique to anticipate the placement of a substance, disposing securing member 34 within hole 18 of planar surface 12, releasably attaching securing member 34 to template grid 10, positioning delivery member 24 so that at least a portion of delivery member 24 is within the patient adjacent the area of interest, releasably attaching delivery member 24 to template grid 10 with securing member 34, and delivering or implanting the substance to a location proximate the area of interest by passing the substance through delivery member 24 and through hole 18. In some embodiments, this method may be altered by substituting alternatives structures and steps as described herein. For example, the method may include releasably attaching a second securing member 34 (and/or delivery member 24) to planar surface 12, wherein the first and second delivery members 24 can be actuated, controlled, manipulated, or otherwise moved independently of one another.

FIG. 8 is a plan view of template grid 10 for use in gynecological implantation. Template grid 10 can be attached to area of interest 74, in this example adjacent to abdomen 76 of patient 78 positioned on table 80. Although FIG. 8 illustrates the use of template grid 10 for implantation adjacent abdomen 76, it can be appreciated that template grid 10 can be used to deliver and/or implant a substance to essentially any part of the body.

Numerous advantages of the invention covered by this document have been set forth in the foregoing description. It will be understood, however, that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A method of implanting a substance in a patient, comprising the steps of:
providing a template grid comprising a planar surface including a first hole;
placing a patient in a position appropriate for an intended medical procedure;
generating images of an area of interest of the patient using an imaging technique;
positioning the template grid adjacent to the area of interest;
aligning the template grid with the images to anticipate the placement of a substance;
disposing a securing member within the first hole;
releasably attaching the securing member to the template grid;
releasably attaching a delivery member to the template grid with the securing member; and
delivering the substance to a location proximate the area of interest by passing the substance through the delivery member.

2. The method of claim 1, wherein the planar surface includes a second hole, and further comprising the step of releasably attaching a second securing member to the second hole.

3. The method of claim 2, further comprising the step of releasably attaching a second delivery member to the template grid with the second securing member.

4. The method of claim 1, wherein the step of placing a patient in a position appropriate for an intended medical procedure includes placing a patient in a position for a diagnostic procedure.

5. The method of claim 1, wherein the step of placing a patient in a position appropriate for an intended medical procedure includes placing a patient in a position for a therapeutic procedure.

6. The method of claim 1, wherein the step of delivering the substance includes delivering a chemotherapeutic drug to a location proximate the area of interest.

7. The method of claim 6, wherein the step of delivering a chemotherapeutic drug includes injecting the chemotherapeutic drug to a location proximate the area of interest with a pump.

8. The method of claim 1, wherein the step of delivering the substance includes delivering a radioactive substance to a location proximate the area of interest.

9. The method of claim 1, wherein the step of generating images of an area of interest of the patient using an imaging technique includes imaging the area of interest by magnetic resonance imaging.

10. The method of claim 1, wherein the step of generating images of an area of interest of the patient using an imaging technique includes imaging the area of interest with infrared imaging.

11. The method of claim 1, wherein the step of aligning the template grid with the images to anticipate the placement of a substance includes coordinating the position of the template grid with the images by using one or more infrared reflecting spheres coupled to the template grid.

12. The method of claim 1, wherein the step of aligning the template grid with the images to anticipate the placement of a substance includes coordinating the position of the template grid with the images by using one or more light emitting diodes coupled to the template grid.

13. The method of claim 1, wherein the step of delivering the substance to a location proximate the area of interest by passing the substance through the delivery member includes passing the substance through a tube.

14. The method of claim 1, wherein the step of delivering the substance to a location proximate the area of interest by passing the substance through the delivery member includes passing the substance through a needle.

15. The method of claim 1, further comprising the step of verifying placement of the substance with an imaging device.

16. The method of claim 1, further comprising the step of configuring the delivery member so that it passes through the first hole and so that at least a portion of the delivery member is positioned adjacent the area of interest.

17. A template grid for implanting a substance into a patient, comprising:
a generally planar surface, wherein the planar surface defines a plurality of holes including a first hole and a second hole;
a securing member disposed in the first hole and releasably attached to the generally planar surface;
a delivery member disposed in the first hole and releasably attached to the generally planar surface by the securing member; and
wherein the delivery member is adapted and configured for delivery of a substance to an area of interest in a patient.

18. The template grid of claim 17, further comprising a second delivery member disposed in the second hole and releasably attached to the generally planar surface by a second securing member.

19. The template grid of claim 18, wherein the first delivery member is releasably attachable to the generally planar surface independently of the second delivery member.

20. The template grid of claim 19, wherein the substance comprises a diagnostic agent.

21. The template grid of claim 17, wherein the substance comprises a chemotherapeutic drug.

22. The template grid of claim 17, wherein the substance is in a slow release form.

23. The template grid of claim 17, wherein the substance is a liquid form.

24. The template grid of claim 17, wherein the substance includes a radioactive seed.

25. The template grid of claim 17, further comprising one or more reference devices coupled to the template grid.

26. The template grid of claim 25, wherein the reference devices include infrared reflecting spheres coupled to the template grid.

27. The template grid of claim 26, wherein the infrared reflecting spheres are coupled to planar surface of the template grid.

28. The template grid of claim 26, wherein the infrared reflecting spheres are coupled to a top surface of the template grid.

29. A method of interstitial delivery of a substance into a patient, comprising the steps of:
providing a template grid comprising a planar surface that includes plurality of holes including a first hole;
positioning the template grid adjacent to an area of interest of a patient, wherein the area of interest is located proximate to an interstitial target location;
aligning the template grid with the area of interest by using an imaging technique;
disposing a securing member within the first hole and releasably attaching the securing member to the template grid;
configuring a delivery member so that it passes through the first hole and so that at least a portion of the delivery member is positioned adjacent the interstitial target location;
releasably attaching the delivery member to the template grid with the securing member; and
delivering a substance to the interstitial location by passing the substance through the delivery member.

30. The method of claim 29, wherein the substance comprises oligonucleotides, peptides, enzymes, hormones, anti-bodies, tyrosine kinase agents, anti-tyrosine kinase agents, anti-sense RNA, growth factors, growth factor inhibitors, anti-angiogenic factors, antibiotics, anesthetics, anti-viral agents, anti-cancer substances, chemotherapeutic agents, anti-proliferative agents, corticosteroids, non-steroidal anti-inflammatory substances, contrast agents, hyaluronic acid, vitamins, methylene blue, diagnostic radioactive substances, technetium 99 m, 18-flurodeoxyglucose, amino levulinic acid, substances used for photodynamic therapy, substances for performing local or regional oxidation or reduction, an infrared antenna, a waveguide, a radiofrequency antenna, a microwave antenna, an ultrasound device, a cryotherapy probe, or mixtures, combinations, or derivatives thereof.

31. The method of claim 29, wherein the substance is in a slow release form.

32. The method of claim 29, wherein the interstitial location comprise the prostate, uterus, ovary, head of the femur, liver, brain, heart, muscle, adipose tissue, or a tumor.

33. A method of interstitial delivery of a substance into a patient, comprising the steps of:
providing a template grid comprising a planar surface that includes plurality of holes including a first hole;
positioning the template grid adjacent to an area of interest of a patient, wherein the area of interest is located proximate to an interstitial target location;
aligning the template grid with the area of interest by using an imaging technique;
coupling a delivery member to the template grid adjacent the first hole so that at least a portion of the delivery member is disposed adjacent the interstitial target location; and
delivering a substance to the interstitial location by passing the substance through the delivery member and through the first hole within the template grid.

34. The method of claim 33, wherein the template grid includes one or more reference devices and wherein the step of aligning the template grid with the area of interest by using an imaging technique includes coordinating the one or more reference devices with the imaging technique.

35. The method of claim 33, wherein the substance comprises oligonucleotides, peptides, enzymes, hormones, anti-bodies, tyrosine kinase agents, anti-tyrosine kinase agents, anti-sense RNA, growth factors, growth factor inhibitors, anti-angiogenic factors, antibiotics, anesthetics, anti-viral agents, anti-cancer substances, chemotherapeutic agents, anti-proliferative agents, corticosteroids, non-steroidal anti-inflammatory substances, contrast agents, hyaluronic acid, vitamins, methylene blue, diagnostic radioactive substances, technetium 99 m, 18-flurodeoxyglucose, amino levulinic acid, substances used for photodynamic therapy, substances for performing local or regional oxidation or reduction, an infrared antenna, a waveguide, a radiofrequency antenna, a microwave antenna, an ultrasound device, a cryotherapy probe, or mixtures, combinations, or derivatives thereof.

36. The method of claim 33, wherein the substance is in a slow release form.

37. The method of claim 33, wherein the interstitial location comprise the prostate, uterus, ovary, head of the femur, liver, brain, heart, muscle, adipose tissue, or a tumor.

38. A template grid for delivering and/or implanting a substance into a patient, comprising:
a generally planar surface, wherein the planar surface defines a plurality of holes including a first hole and a second hole;
a delivery member disposed in the first hole and releasably attached to the generally planar surface;
wherein the delivery member is adapted and configured for delivery of a substance to an area of interest in a patient; and
a reference device coupled to the template grid.

39. A device for delivering and/or implanting a substance into a patient, comprising:
a template grid having a top surface and a generally planar surface;
wherein the planar surface defines one or more holes including a first hole;
wherein the top surface defines a third hole;
a first reference device coupled to the template grid adjacent the first hole; and
a second reference device coupled to the template grid adjacent the second hole.
